(19) United States
(12) Patent Application Publication
(10) Pub. No.: US 2026/0007456 A1
(43) Pub. Date: Jan. 8, 2026

(54) PUNCTURE DEVICE HAVING AN EXTENDABLE CONDUCTIVE MEMBER AND INSULATIVE LINER

(71) Applicant: Boston Scientific Scimed, Inc., Maple Grove, MN (US)

(72) Inventors: Lauren Koon, Etobicoke (CA); Christian Balkovec, Cambridge (CA)

(21) Appl. No.: 19/258,682

(22) Filed: Jul. 2, 2025

Related U.S. Application Data
(60) Provisional application No. 63/668,111, filed on Jul. 5, 2024.

(51) Int. Cl. A61B 18/14; A61B 18/00

(52) U.S. Cl. CPC A61B 18/1477; A61B 2018/00077; A61B 2018/00083; A61B 2018/00184; A61B 2018/00351; A61B 2018/1475

(57) ABSTRACT

A device for puncturing a tissue within a heart is disclosed. The device includes a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires. The device further includes a conductive member longitudinally extending from the hollow delivery lumen of the needle, the conductive member having a proximal end and an opposite distal end portion that is electrically conductive and terminates at a functional tip. When the conductive member perforates the tissue, the needle is proximate the conductive member and the needle is not in contact with the tissue.

### CROSS REFERENCE TO RELATED APPLICATIONS

The present application claims priority to U.S. Provisional Application No. 63/668,111, filed July 5, 2024, the entire disclosure of which is incorporated herewith in its entirety.

### TECHNICAL FIELD

The present disclosure relates to medical systems and methods for puncturing a tissue within a heart of a patient. More specifically, the present disclosure relates to a perforation device having a mechanical needle and a conductive member in which the device reduces coring when perforating a tissue.

### BACKGROUND

In general, in medical procedures within a heart of a patient, when attempting to puncture a tissue with a mechanical needle defining a hollow lumen, occasionally difficulty is encountered whereby the mechanical force applied is not sufficient to facilitate the needle piercing through the tissue. In these circumstances, a physician may choose to transmit electrical current, e.g., radiofrequency energy, through the mechanical needle via an electrocautery system or other electrical generator systems. By transmitting sufficient electrical current though the mechanical needle, the desired tissue to be punctured is heated and vaporized, thereby creating a puncture hole through the tissue. With this, the tissue at the hollow center of the needle tip is not vaporized, but rather the closed electrical pathway surrounding the needle tip separates the portion of the tissue from the rest of the tissue wall. Thus, the electrified hollow mechanical needle acts as a hole punch and separates a piece of tissue from the tissue wall.

A problem with this approach is that when puncturing the tissue, the hollow mechanical needle creates a core of tissue that may be released into the bloodstream. Releasing a piece of tissue into the bloodstream creates a risk of embolization. However, it would also still be advantageous to have a puncture needle having a hollow lumen to facilitate in fluid injections, aspiration, guidewire, or other medical device insertions, among other benefits.

Furthermore, in circumstances in which a conductive member delivers electrical current in a liquid environment, the conductive member generally disperses current from all its conductive surfaces. Generally, current is delivered to a conductive medium via a conductive member. When two different media are present, for example tissue and blood, and there is a desire to selectively deliver current to one media over the other, current leakage into the second medium must be minimized. Thus, there is a need to control the surface area of a conductive surface intended to deliver current to a conductive medium. Additionally, for conductive members with a hollow lumen, as discussed above, a conductive accessory device inside of the hollow lumen may become inadvertently electrified when extending through the hollow lumen, further dispersing current and potentially becoming a safety hazard for the physician.

Against this background, there exists a continuing need in the industry to provide improved devices and methods for gaining access to and perforating a tissue within a patient's heart. An object of the present invention is therefore to provide such an apparatus.

### SUMMARY

In Example 1, a device for puncturing a tissue within a heart includes a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires. The device also includes a conductive member longitudinally extending from the hollow delivery lumen of the needle, the conductive member having a proximal end and an opposite distal end portion that is electrically conductive and terminates at a functional tip; wherein when the conductive member perforates the tissue, the needle is proximate the conductive member and the needle is not in contact with the tissue.

Example 2 is the device of Example 1 wherein the conductive member is a radiofrequency perforation device.

Example 3 is the device of Example 1 wherein when perforating the tissue the conductive member is at a length that prevents the hollow lumen of the needle from being in contact with the tissue, and wherein the device reduces tissue coring when perforating the tissue.

Example 4 is the device of Example 1 wherein the conductive member does not define a lumen.

Example 5 is the device of Example 1 further comprising an advancement mechanism.

Example 6 is the device of Example 5 wherein the advancement mechanism is configured to interact with the conductive member, and wherein the advancement mechanism longitudinally advances the conductive member from the hollow delivery lumen of the needle.

Example 7 is the device of Example 5 wherein the advancement mechanism is an accessory device.

Example 8 is the device of Example 1 further including a dilator comprising an elongated body having a body length, a proximal end and a distal end portion terminating in a distal tip and defining a dilator lumen extending through the elongate body.

Example 9 is the device of any of Examples 1-8 wherein the needle and conductive member are advanced through the dilator lumen, and wherein only the conductive member protrudes from the distal tip of the dilator during perforation of the tissue.

Example 10 is the device of Example 1 wherein the needle further includes a secondary lumen that is dimensioned to slidingly receive at least one or more secondary wires.

Example 11 is the device of any of Examples 1-10 wherein the conductive member is longitudinally advanced through the secondary lumen of the needle.

Example 12 is the device of Example 1 wherein the conductive member further includes an insulative layer along a portion of the conductive member, and wherein the insulative layer minimizes electric current leakage.

Example 13 is the device of Example 12 wherein the insulative layer stops short of the distal end portion of the conductive member such that the distal end of the conductive member is electrically exposed.

Example 14 is the device of Example 1 wherein an insulative layer is positioned along a portion of an exterior surface of the needle.

Example 15 is the device of Example 1 wherein an insulative layer is positioned along the delivery lumen of the needle.

In Example 16, a device for puncturing a tissue within a heart includes a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires. The device also includes a conductive member longitudinally extending from the hollow delivery lumen of the needle, the conductive member having a proximal end and an opposite distal end portion that is electrically conductive and terminates at a functional tip; wherein when the conductive member perforates the tissue, the needle is proximate the conductive member and the needle is not in contact with the tissue.

Example 17 is the device of Example 16 wherein the conductive member is a radiofrequency perforation device, and wherein the conductive member does not define a lumen.

Example 18 is the device of Example 16 wherein when perforating the tissue the conductive member is at a length that prevents the hollow lumen of the needle from being in contact with the tissue, and wherein the device reduces tissue coring when perforating the tissue.

Example 19 is the device of Example 16 further comprising an advancement mechanism.

Example 20 is the device of Example 19 wherein the advancement mechanism is configured to interact with the conductive member, and wherein the advancement mechanism longitudinally advances the conductive member from the hollow delivery lumen of the needle.

Example 21 is the device of Example 19 wherein the advancement mechanism is an accessory device.

Example 22 is the device of Example 16 further including a dilator comprising an elongated body having a body length, a proximal end and a distal end portion terminating in a distal tip and defining a dilator lumen extending through the elongate body, wherein the needle and conductive member are advanced through the dilator lumen, and wherein only the conductive member protrudes from the distal tip of the dilator during perforation of the tissue.

Example 23 is the device of Example 16 wherein the needle further includes a secondary lumen that is dimensioned to slidingly receive at least one or more secondary wires, and wherein the conductive member is longitudinally advanced through the secondary lumen of the needle.

Example 24 is the device of Example 16 wherein the conductive member further includes an insulative layer along a portion of the conductive member, and wherein the insulative layer minimizes electric current leakage.

Example 25 is the device of Example 24 wherein the insulative layer stops short of the distal end portion of the conductive member such that the distal end of the conductive member is electrically exposed.

Example 26 is the device of Example 16 wherein an insulative layer is positioned along a portion of an exterior surface of the needle.

Example 27 is the device of Example 16 wherein an insulative layer is positioned along the delivery lumen of the needle.

In Example 28, a system for puncturing a tissue within a heart includes a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires. The system also includes a conductive member longitudinally extending from the hollow delivery lumen of the needle, the conductive member having a proximal end and an opposite distal end portion that is electrically conductive and terminates at a functional tip, wherein when the conductive member perforates the tissue, the needle is proximate the conductive member and the needle is not in contact with the tissue. The system further includes an advancement mechanism configured to interact with the conductive member and longitudinally advance the conductive member from the hollow delivery lumen of the needle.

Example 29 is the system of Example 28 wherein the conductive member is a radiofrequency perforation device, and wherein the conductive member does not define a lumen.

Example 30 is the system of Example 28 wherein when perforating the tissue the conductive member is at a length that prevents the hollow lumen of the needle from being in contact with the tissue, and wherein the device reduces tissue coring when perforating the tissue.

Example 31 is the system of Example 28 wherein the advancement mechanism is an accessory device.

Example 32 is the system of Example 28 further including a dilator comprising an elongated body having a body length, a proximal end and a distal end portion terminating in a distal tip and defining a dilator lumen extending through the elongate body, wherein the needle and conductive member are advanced through the dilator lumen, and wherein only the conductive member protrudes from the distal tip of the dilator during perforation of the tissue.

Example 33 is the system of Example 28 wherein the needle further includes a secondary lumen that is dimensioned to slidingly receive at least one or more secondary wires, and wherein the conductive member is longitudinally advanced through the secondary lumen of the needle.

In Example 34, a method for puncturing a tissue within a heart includes providing a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires. The method for puncturing a tissue within a heart further includes advancing a conductive member longitudinally extending from the hollow delivery lumen of the needle, the conductive member having a proximal end and an opposite distal end portion that is electrically conductive and terminates at a functional tip; wherein when the conductive member perforates the tissue, the needle is proximate the conductive member and the needle is not in contact with the tissue.

Example 35 is the method of Example 34 further including providing an insulative layer along a portion of the conductive member.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIGS. 1A-1B are schematic illustrations of a medical procedure within a patient's heart for gaining access to the left atrium as well as the epicardial space, according to embodiments of the present disclosure.

FIGS. 2A-2C are schematic illustrations of a device for puncturing a tissue within a heart comprising a mechanical needle and a conductive member, according to embodiments of the present disclosure.

FIG. 3 is an illustration of the device of FIGS. 2A-2C longitudinally advanced through a dilator, according to embodiments of the present disclosure.

FIGS. 4A-4C are schematic illustrations of the device of FIGS. 2A-2C having an advancement mechanism for selectively advancing the conductive member, according to embodiments of the present disclosure.

FIGS. 5A-5B are schematic illustrations of the device of FIGS. 2A-2C the needle having a primary lumen and a secondary lumen, according to embodiments of the present disclosure.

FIGS. 6A-6B are schematic illustrations of the device of FIGS. 2A-2C having an insulative layer along a portion of the device to insulate a portion of the conductive member, according to embodiments of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

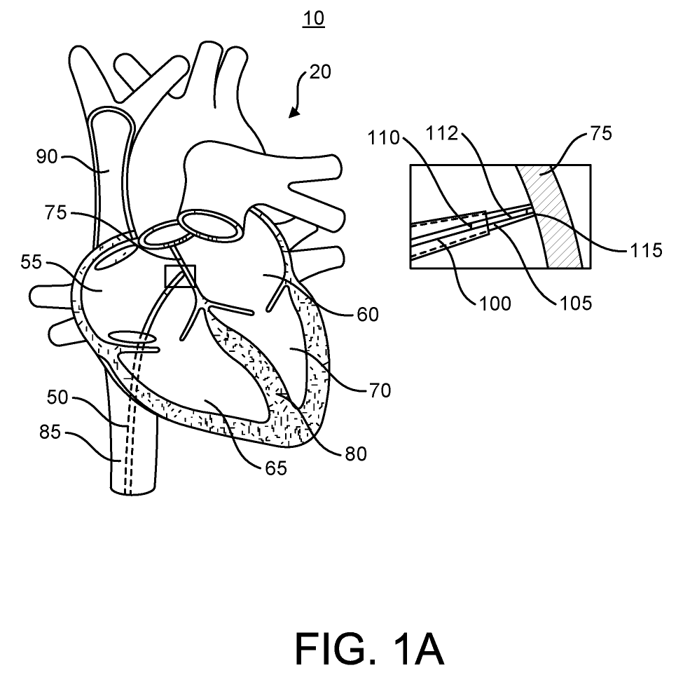

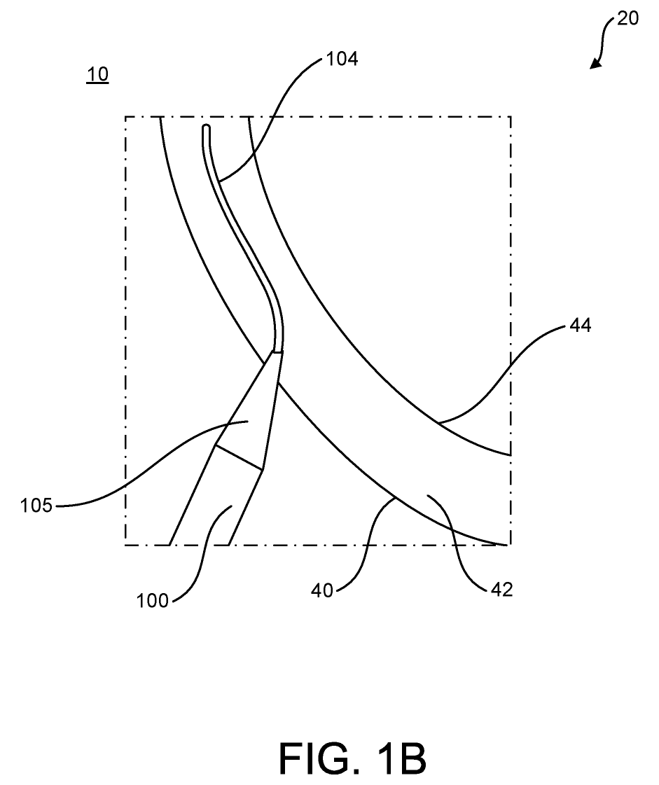

FIGS. 1A and 1B are schematic illustrations of medical procedures within a patient's heart for gaining transseptal access as well as access to the epicardial space by puncturing tissue within the heart, according to embodiments of the present disclosure. FIG. 1A is an illustration of a medical procedure 10 within a patient's heart 20 utilizing a transseptal access system 50. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

Procedures for providing access to the left atrium 60 use transseptal access systems and devices for subsequent deployment of the aforementioned diagnostic and/or therapeutic devices within the left atrium 60. In these procedures, a target tissue site can be defined by tissue on the atrial septum 75. The target site is accessed via the inferior vena cava (IVC) 85, for example through the femoral vein, according to conventional catheterization techniques. In other embodiments, access to the target site on the atrial septum 75 may be accomplished using a superior approach wherein the transseptal access system 50 is advanced into the right atrium 55 via the superior vena cava (SVC) 90.

Transseptal access system procedures may include many devices like an introducer sheath 100, a dilator 105, a puncture device 110 having a distal end portion 112 terminating in a distal tip 115, and a guidewire. In various embodiments, the puncture device 110 is a mechanical puncture device (e.g., a needle). In other embodiments, the puncture device 110 may be an RF perforation device. In still other embodiments, as will be discussed in greater detail below, the puncture device 110 may be a mechanical needle that a user may choose to then transmit electrical current through. In an embodiment, the puncture device 110 can be disposed within the dilator 105, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. In an embodiment, the dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20.

When the guidewire, sheath 100 and dilator 105 have been positioned in the SVC 90, the guidewire is removed from the body, and the sheath 100 and the dilator 105 are retracted so that their distal ends are positioned in the right atrium 55. In an embodiment, the puncture device 110 described can then be introduced into the dilator 105, and advanced toward the heart 20. In other embodiments, the puncture device 110 described may be introduced prior to the retraction of the sheath 100 and the dilator 105 from the SVC into the right atrium 55. The puncture device 110 is then positioned such that the tip 115 is aligned with or protruding slightly from the distal end of the dilator 105.

In embodiments where the puncture device 110 is an RF perforation device with the tip electrode 115 and dilator 105 positioned at the target site, or in embodiments in which the puncture device 110 is a mechanical needle that a user may choose to then transmit electrical current through with the tip 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least 200 V RMS, or in certain embodiments about 565 V (peak- to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 0.1 second and about 5 seconds. In other embodiments, the step of delivering energy occurs over a period of about 300 milliseconds.

Still another medical procedure 10 developed for diagnosing or treating physiological ailments originating within a heart 20 includes epicardial ablation to help restore a regular heart rhythm, as shown in FIG. 1B. As illustrated, the heart 20 includes a pericardium 40, a pericardial cavity 42 and a myocardium 44. The heart 20 is typically approached using a subxiphoid approach. Epicardial access is achieved via puncturing a layer of the pericardium 40 while avoiding the myocardium 44 of the heart. The pericardium 40 is a tough, double-walled, fibroelastic sac encompassing the heart 20 and the roots of the great vessels. The pericardium 40 includes two layers, an outer layer made of strong connective tissue often referred to as the fibrous pericardium, and an inner layer made of serous membrane often referred to as the serous pericardium. The mesothelium, or mesothelial cells, that constitutes the serous pericardium also covers the myocardium of the heart as epicardium, resulting in a continuous serous membrane invaginated onto itself as two opposing surfaces such as over the fibrous pericardium 40 and over the heart 20. This creates a pouch-like virtual or potential space around the heart 20 enclosed between the two opposing serosal surfaces, often referred to as the pericardial space or pericardial cavity 42.

In some embodiments, the pericardium 40 may be punctured with a puncture device 110. In various embodiments, the puncture device 110 is a mechanical puncture device (e.g., a mechanical needle). In other embodiments, the puncture device 110 may be an RF perforation device. In still other embodiments, as will be discussed in greater detail below, the puncture device 110 may be a mechanical needle that a user may choose to then transmit electrical current through. Once punctured, a dilator 105 is advanced to dilate the puncture created by the needle through the pericardium 40. In certain embodiments, a sheath 100 may be advanced with the dilator 105 simultaneously. In other embodiments, the sheath 100 may be advanced afterwards. In an embodiment, the sheath 100 and the dilator 105 may then be withdrawn to leave the guidewire 104 in the pericardial cavity 42. Minimally invasive access to the epicardium is required for diagnosis and treatment of a variety of arrhythmias and other conditions. During epicardial ablation, tiny scars are created on the outside of the heart to create a transmural lesion. In other words, to achieve an ablated tissue through the thick muscle of the heart.

The present disclosure describes novel systems and methods for providing safe access to the heart. As will be explained in greater detail herein, the embodiments of the present disclosure improve systems and methods for puncturing a tissue in ways that will reduce tissue coring when perforating the tissue.

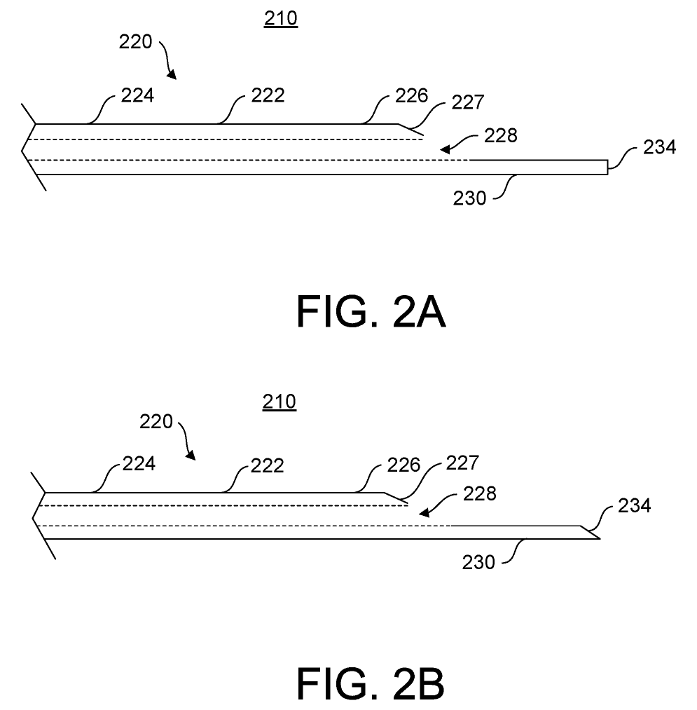

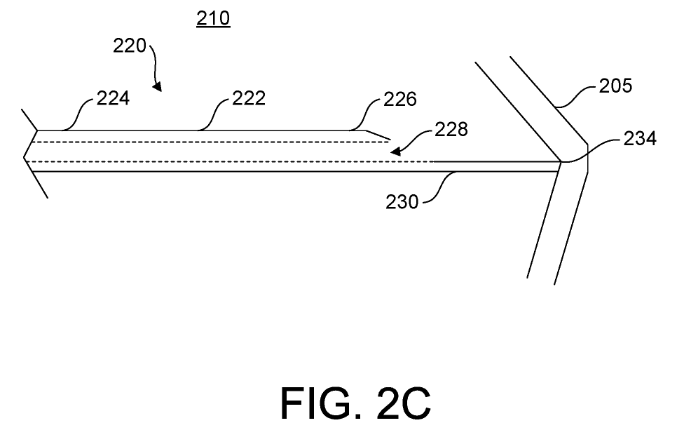

FIGS. 2A-2C are schematic illustrations of a device 210 for puncturing a tissue 205 within a heart of a patient comprising a mechanical needle 220 and a conductive member 230 according to an embodiment of the present disclosure. As shown, the device includes the mechanical needle 220 having an elongated body 222, a proximal end portion 224 and an opposite distal end portion 226 terminating in a distal tip 227. Additionally, the needle 220 defines a hollow delivery lumen 228 extending through the elongate body 222 and dimensioned to slidingly receive and allow longitudinal translation of at least one or more wires. In an embodiment, the at least one or more wires may include a piercing stylet, a guidewire, among other secondary medical devices. In an embodiment, the hollow lumen 228 may facilitate in fluid injections, aspiration, or other medical device insertions. As shown, in FIGS. 2A-2C, the device 210 may further include a conductive member 230 longitudinally extending from the hollow delivery lumen 228 of the needle 220. The conductive member 230 includes a proximal end portion and an opposite distal end portion that is electrically conductive and terminates at a functional tip 234. In an embodiment, the conductive member is a radiofrequency perforation device. In an embodiment, as shown, the conductive member 230 does not define its own lumen.

In an embodiment, the proximal portion of the conductive member 230 can be handled directly by the user when the conductive member 230 is energized. In some embodiments, the proximal portion is of a unitary construction formed entirely of an electrically insulative material. In certain embodiments, one exemplary class of materials for construction of the proximal portion can include various grades of polytetrafluoroethylene (PTFE), polyetheretherketone (PEEK), among others. In some embodiments, the proximal portion can further include reinforcing elements, e.g., a polymeric braid or coil, to enhance the structural properties, e.g., stiffness, torque transfer capability, and the like. In some embodiments, the proximal portion is formed of a metal (e.g., a metal hypotube), and includes an outer electrically insulating layer, as will be discussed in greater detail below.

In certain embodiments, the distal portion of the conductive member 230 is electrically conductive and is capable of transferring radiofrequency energy supplied by an external RF generator to the functional tip 234 for subsequent delivery to the target tissue 205. Any biocompatible electrically conductive material may be selected for construction of the distal portion of the conductive member 230. Exemplary materials may include stainless steel, nickel- titanium alloy, and the like. Further, for ease of illustration, the distal portion is depicted in FIGS. 2A-2C as a single solid structure, although the construction of the distal portion of the conductive member 230 can vary to accommodate the particular structural requirements for the perforation device 210, as will be further explained below. For example, in some embodiments, the distal portion of the conductive member 230 can be constructed as a solid rod, a tube or a coil. Additionally, in certain embodiments, the distal portion of the conductive member 230 can be constructed in multiple segments, e.g., a solid rod or hypotube in the regions nearest the proximal portion, and a coiled structure more distally to provide enhanced flexibility and torqueability. In some embodiments, the distal portion of the conductive member 230 can have a composite construction, e.g., a solid or tubular core conductor surrounded by a wire coil.

In certain embodiments, once a physician determines that the mechanical needle 220 will not pierce through a tissue for any reason, or in general determines the need to perforate a tissue with electrical current, the physician may advance the conductive member 230 from the hollow delivery lumen 228 of the mechanical needle 220 and position the conductive member 230 at the tissue 205. In an embodiment, energy may be delivered from an energy source, e.g., an RF generator, through the conductive member 230 to the tip electrode 234 and the tissue 205, thus perforating the tissue. In an embodiment, as shown in FIG. 2B, the functional tip 234 may also be sharp to further facilitate the puncture process before or as the physician attempts to electrify the conductive member 230.

In an embodiment, as shown in FIG. 2C, when the functional tip 234 of the conductive member 230 is perforating the tissue 205, the distal portion 226 of the needle 220 is proximate the conductive member and so the needle 220, and thus the hollow lumen 228 of the needle 220, are not in contact with the tissue 205. In some embodiments, when perforating the tissue 205 the conductive member 230 is at a length proximate the needle 220 that prevents the hollow lumen 228 of the needle 220 from being in contact with the tissue 205. In an embodiment, when perforating the tissue, the conductive member 230 is at a length that prevents substantial coverage of the hollow lumen 328 of the needle 220 by tissue 205, thereby preventing the formation of a tissue core when electrical current is transmitted through the mechanical needle 220 to the distal functional tip 234 of the conductive member 230. Thus, in certain embodiments, the device 210 reduces tissue coring when perforating the tissue 205 because only the conductive member 230, which does not include its own lumen, is in contact with the tissue 205 when the needle 220 is electrified and as the tissue 205 is being perforated and the hollow lumen 228 of the needle 220 is at a safe distance away from the perforation so that a piece of tissue 205 is not separated from the tissue wall 205 but rather the conductive member 230 perforates through the tissue 205 at the target site. Therefore, in an embodiment, the device 210 reduces the risk of coring and the risk of medical complications, like embolization.

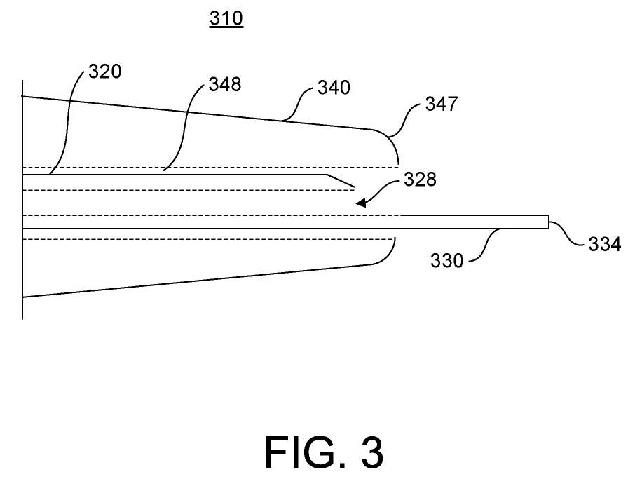

FIG. 3 is an illustration of a device 310 further comprising a dilator 340 according to an embodiment of the present disclosure. The device 310 may be substantially structurally and functionally identical to the device 210 of FIGS. 2A-2C, except as described in connection with FIG. 3. In an embodiment, the dilator 340 may include an elongated body having a body length, a proximal end and a distal end portion terminating in a distal tip 347 and defining a dilator lumen 348 extending through the elongate body of the dilator 340. In an embodiment, as shown in FIG. 3, the needle 320 and conductive member 330 are advanced through the dilator lumen 348 of the dilator 340. In certain embodiments, only the conductive member 330, and thus the distal tip 334 of the conductive member 330, protrudes from the distal tip 347 of the dilator 340 during perforation of the tissue. In certain embodiments, the hollow lumen 348 of the dilator 340 may itself be insulated. In an embodiment, when used with the insulative dilator 340, the conductive member 330 may be the only portion of the mechanical needle 320 that is extended from the lumen 348 of the dilator 340 during puncture. In certain embodiments, this ensures that the hollow lumen 328 of the needle 320 is not in contact with the tissue when perforated.

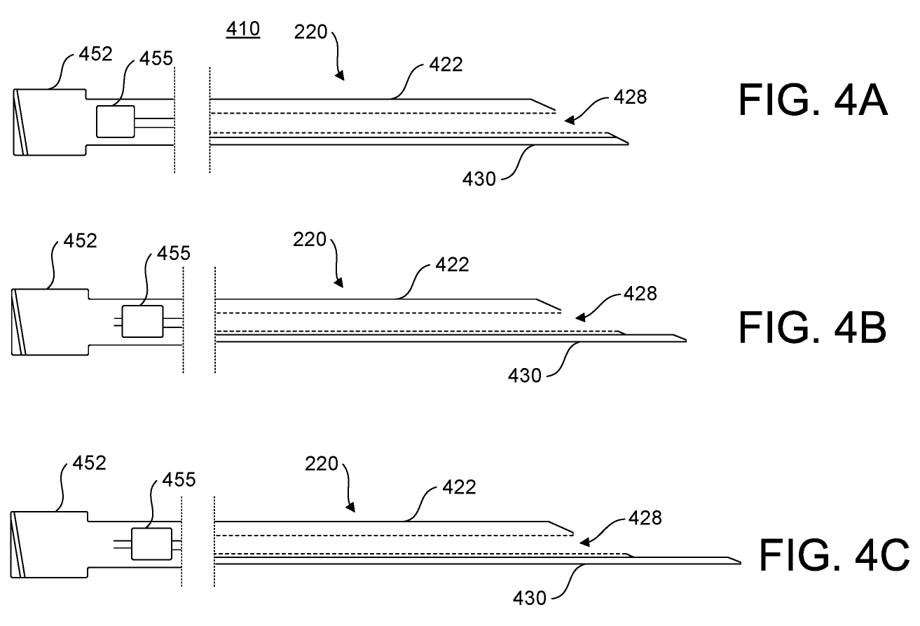

FIGS. 4A-4C are schematic illustrations of a device 410 for puncturing a tissue within a heart having an advancement mechanism 455 according to an embodiment of the present disclosure. The device 410 may be substantially structurally and functionally identical to the device 210, 310 of FIGS. 2A-3, except as described in connection with FIGS. 4A-4C. In an embodiment, the device 410 may include the advancement mechanism 455 configured to interact with the conductive member 430. In an embodiment, as shown, the advancement mechanism 455 longitudinally selectively advances the conductive member 430 from the hollow delivery lumen 428 of the needle 420. In some embodiments, the conductive member 430 may advance from a secondary lumen that is not the hollow delivery lumen 428. In an embodiment, this ensures that the hollow delivery lumen 428 can accommodate other additional accessory devices. In an embodiment, the advancement mechanism 455 is positioned on the handle 452 of the needle 420 which enables extension of the conductive member 430. In other embodiments, the advancement mechanism 455 may be positioned on a handle of a dilator, as discussed above. In an embodiment, the advancement mechanism 455 may be an accessory device that may be attached to the handle of the needle or the handle of the dilator when desired to selectively extend the conductive member from the tip of the hollow lumen 428 of the mechanical needle 420.

In some embodiments, the advancement mechanism 455 may be a knob, a dial, a screw, a collar or any other structure known in the art that may guide in advancing the conductive member 430. In some embodiments, the conductive member 430 may include at least one or more grooves located on the body of the conductive member 430. Additionally, in some embodiments, the advancement mechanism 455 may be a knob, or a screw, having a top portion that protrudes from the handle 452, so that a user may have access to the advancement mechanism 455, and a bottom portion positioned through the handle 452 and within the lumen. In certain embodiments, when the knob aligns with one of the at least one or more grooves within the conductive member 430, a user may longitudinally advance the top portion of the knob across the handle 452 thereby advancing the conductive member 430 longitudinally within the hollow lumen 428 of the needle 420. Thus, in one embodiment, the conductive member 430 is selectively advanced by the advancement mechanism 455 on the handle 452 of the needle 420 or dilator. In other embodiments, the advancement mechanism 455 may be a dial having a traction element, a top portion protruding from the handle 452 that is accessible to the user and a bottom portion positioned through the handle 452 and within the lumen 428 having access to the conductive member 430. In certain embodiments, the friction applied by the dial to the conductive member 430 may advance the conductive member 430 longitudinally from the lumen 428, or a secondary lumen. Still other configurations known in the art may be contemplated to selectively advance the advancement mechanism 455 and thereby advancing the conductive member 430. In certain embodiments, the advancement mechanism 455 may be an accessory device that can be connected to the handle 452 to aid in advancing the conductive member 430.

In certain embodiments, the advancement mechanism 455 may further include an indicator mechanism (not shown) positioned on the handle 452. In other embodiments, the indicator mechanism may be positioned on the dilator body (not shown). In an embodiment, the indicator mechanism indicates a protrusion length of the conductive member 430. Thus, in certain embodiments, the indicator mechanism may provide a user with feedback regarding the protrusion length of the conductive member 430 from the hollow lumen 428 of the needle 420. In some embodiments, the protrusion length is measured from the distal tip of the needle 420 to the distal tip of the conductive member 430. In some embodiments, the indicator mechanism may indicate the length of the conductive member 430 from the distal tip of the needle 420, while in other embodiments, the indicator mechanism may indicate meaningful protrusion points along the conductive member 430.

In certain embodiments, the indicator mechanism includes an indicator that may be a visual, a tactile or an auditory indicator. In certain embodiments, other indicators known in the art that provide communication to the user regarding the protrusion length of the conductive member 430 may be contemplated. By explicitly indicating the amount of protrusion of the conductive member 430 from the needle 420, the user may more precisely tailor the protrusion length of the conductive member 430 to optimize the perforation device method that is being employed.

In one embodiment, the indicator mechanism may include an indicator light that communicates the protrusion length of the conductive member 430. In other embodiments, the indicator mechanism may include at least one or more tactile notches, or raised surface features, that allow a user to feel and/or observe the position of the conductive member 430. In still other embodiments, the indicator mechanism may include auditory feedback, such as clicking sounds, that are created when the user advances the conductive member 430 past certain points within the needle 420. In certain embodiments, each indicator of the indicator mechanism may correspond to one millimeter of advancement by the conductive member 430. In certain embodiments, each indicator of the indicator mechanism may correspond to less than one millimeter of advancement by the conductive member 430. In certain embodiments, each indicator of the indicator mechanism may correspond to two millimeters of advancement by the conductive member 430. In other embodiments, any other application of force or motion may be translated into the lateral movement of the conductive member 430. In still other embodiments, the indicator mechanism may include at least one or more markings on the handle 452 representing the protrusion length of the conductive member 430.

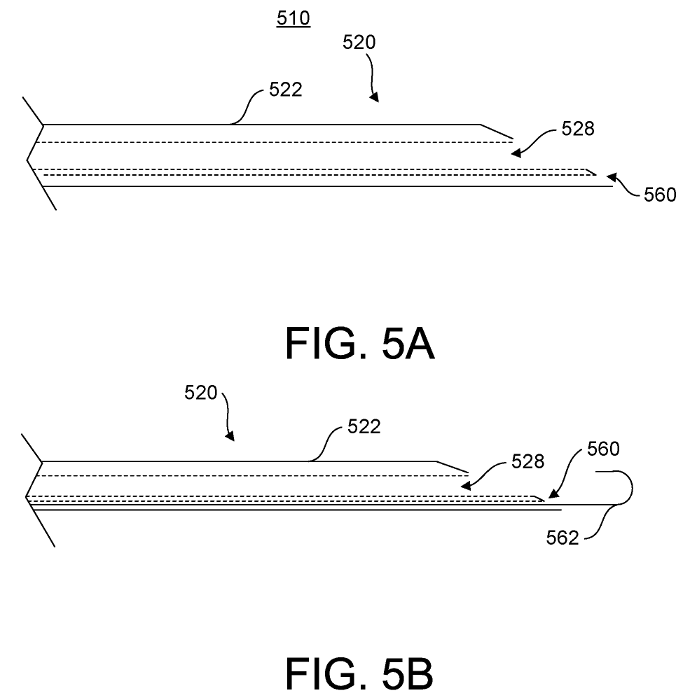

FIGS. 5A-5B illustrate a device 510 for puncturing a tissue within a heart according to an embodiment of the present disclosure. The device 510 may be substantially structurally and functionally identical to the device 210, 310, 410 of FIGS. 2A-4C, except as described in connection with FIGS. 5A-5B. In an embodiment, the mechanical needle 520 may include a secondary lumen 560 that accommodated one or more secondary wires. In an embodiment, the secondary lumen 560 may aid in insertion of secondary devices 562 including a piercing stylet, a guidewire, among others. In an embodiment, as shown, the secondary lumen 560 is a separate feature than the primary delivery lumen 528 of the needle 520. In an embodiment, the conductive member may advance longitudinally through the secondary lumen 560.

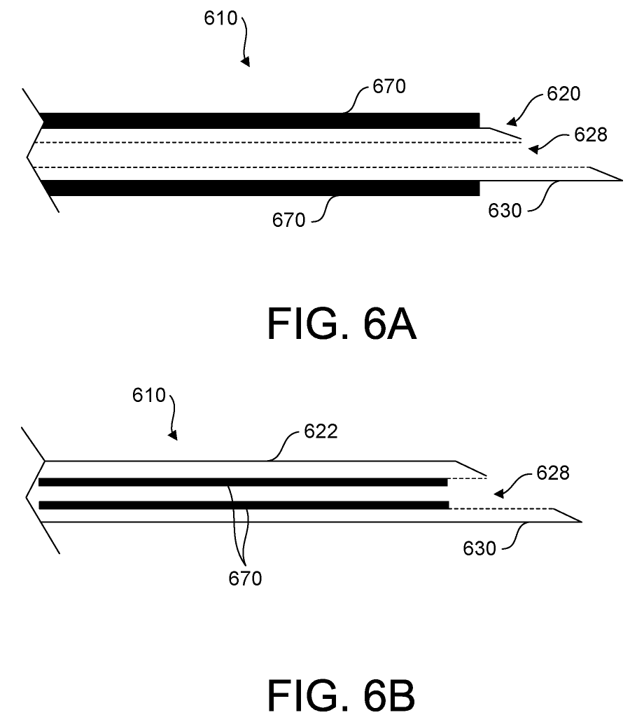

FIGS. 6A-6B illustrate a device 610 for puncturing a tissue within a heart having an insulative layer according to an embodiment of the present disclosure. The device 610 may be substantially structurally and functionally identical to the device 210, 310, 410, 510 of FIGS. 2A- 5B, except as described in connection with FIGS. 6A-6B. In an embodiment, as shown, a portion of the device 610 may include an insulative layer to prevent current leakage from the conductive member 630 when electrified. In some embodiments, this increases the amount of current delivered to the portions of the conductive member 630 that remain uninsulated, as discussed above. In an embodiment, an insulative layer 670 may be along a portion of the conductive member 630 to minimizes electric current leakage. In certain embodiments, the insulative layer 670 stops short of the distal end portion of the conductive member 630 such that the distal end of the conductive member 630 is electrically exposed. In an embodiment, wherein an insulative layer 670 is positioned along a portion of an exterior surface of the needle 620, as shown in FIG. 6A. In other embodiments, an insulative layer 670 is positioned along the delivery lumen 628 of the needle 620. In some embodiments, the insulative layer 670 may be a coating or other dielectric material. In an embodiment, the insulative layer 670 includes as insulative medium such as dextrose, carbon dioxide, oxygen, or other gasses or liquids that do not conduct electrical current.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean "one and only one" unless explicitly so stated, but rather "one or more." Moreover, where a phrase similar to "at least one of A, B, or C" is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms "couples," "coupled," "connected," "attached," and the like along with variations thereof are used to include both arrangements wherein two or more components are in direct physical contact and arrangements wherein the two or more components are not in direct contact with each other (e.g., the components are "coupled" via at least a third component), but still cooperate or interact with each other.

In the detailed description herein, references to "one embodiment," "an embodiment," "an example embodiment," etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A device for puncturing a tissue within a heart, the device comprising:a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires; anda conductive member longitudinally extending from the hollow delivery lumen of the needle, the conductive member having a proximal end and an opposite distal end portion that is electrically conductive and terminates at a functional tip;wherein when the conductive member perforates the tissue, the needle is proximate the conductive member and the needle is not in contact with the tissue.

2. The device of claim 1, wherein the conductive member is a radiofrequency perforation device, and wherein the conductive member does not define a lumen.

3. The device of claim 1, wherein when perforating the tissue the conductive member is at a length that prevents the hollow lumen of the needle from being in contact with the tissue, and wherein the device reduces tissue coring when perforating the tissue.

4. The device of claim 1, further comprising an advancement mechanism.

5. The device of claim 4, wherein the advancement mechanism is configured to interact with the conductive member, and wherein the advancement mechanism longitudinally advances the conductive member from the hollow delivery lumen of the needle.

6. The device of claim 4, wherein the advancement mechanism is an accessory device.

7. The device of claim 1, further including a dilator comprising an elongated body having a body length, a proximal end and a distal end portion terminating in a distal tip and defining a dilator lumen extending through the elongate body, wherein the needle and conductive member are advanced through the dilator lumen, and wherein only the conductive member protrudes from the distal tip of the dilator during perforation of the tissue.

8. The device of claim 1, wherein the needle further includes a secondary lumen that is dimensioned to slidingly receive at least one or more secondary wires, and wherein the conductive member is longitudinally advanced through the secondary lumen of the needle.

9. The device of claim 1, wherein the conductive member further includes an insulative layer along a portion of the conductive member, and wherein the insulative layer minimizes electric current leakage.

10. The device of claim 9, wherein the insulative layer stops short of the distal end portion of the conductive member such that the distal end of the conductive member is electrically exposed.

11. The device of claim 1, wherein an insulative layer is positioned along a portion of an exterior surface of the needle.

12. The device of claim 1, wherein an insulative layer is positioned along the delivery lumen of the needle.

13. A system for puncturing a tissue within a heart, the system comprising:a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires;
a conductive member longitudinally extending from the hollow delivery lumen of the needle, the conductive member having a proximal end and an opposite distal end portion that is electrically conductive and terminates at a functional tip,wherein when the conductive member perforates the tissue, the needle is proximate the conductive member and the needle is not in contact with the tissue; and
an advancement mechanism configured to interact with the conductive member and longitudinally advance the conductive member from the hollow delivery lumen of the needle.

14. The system of claim 13, wherein the conductive member is a radiofrequency perforation device, and wherein the conductive member does not define a lumen.

15. The system of claim 13, wherein when perforating the tissue the conductive member is at a length that prevents the hollow lumen of the needle from being in contact with the tissue, and wherein the device reduces tissue coring when perforating the tissue.

16. The system of claim 13, wherein the advancement mechanism is an accessory device.

17. The system of claim 13, further including a dilator comprising an elongated body having a body length, a proximal end and a distal end portion terminating in a distal tip and defining a dilator lumen extending through the elongate body, wherein the needle and conductive member are advanced through the dilator lumen, and wherein only the conductive member protrudes from the distal tip of the dilator during perforation of the tissue.

18. The system of claim 13, wherein the needle further includes a secondary lumen that is dimensioned to slidingly receive at least one or more secondary wires, and wherein the conductive member is longitudinally advanced through the secondary lumen of the needle.

19. A method for puncturing a tissue within a heart, the method comprising:
providing a mechanical needle having an elongated body, a proximal end and an opposite distal end portion terminating in a distal tip, the needle defining a hollow delivery lumen extending through the elongate body and dimensioned to slidingly receive and allow longitudinal translation of at least one or more wires;and advancing a conductive member longitudinally extending from the hollow delivery lumen of the needle, the conductive member having a proximal end and an opposite distal end portion that is electrically conductive and terminates at a functional tip;
wherein when the conductive member perforates the tissue, the needle is proximate the conductive member and the needle is not in contact with the tissue.

20. The method of claim 19, further includes providing an insulative layer along a portion of the conductive member.
